(19) United States
(12) Patent Application Publication
(10) Pub. No.: US 2024/0148952 A1
(43) Pub. Date: May 9, 2024

(54) Systems and Methods for Hyperthermic Cancer Treatment

(71) Applicant: C. R. Bard, Inc., Franklin Lakes, NJ (US)

(72) Inventors: Michael R. Hoglund, Windsor, CO (US); Marc E. Voorhees, Arvada, CO (US)

(21) Appl. No.: 18/280,176

(22) PCT Filed: Mar. 8, 2022
(86) PCT No.: PCT/US22/19376

§ 371 Date: Sep. 1, 2023

Related U.S. Application Data
(60) Provisional application No. 63/158,263, filed on Mar. 8, 2021.

(51) Int. Cl. A61M 1/36

(52) U.S. Cl. CPC A61M 1/369; A61M 1/3659; A61M 2205/3368; A61M 2205/366

(57) ABSTRACT

Disclosed are systems and methods for hyperthermic cancer treatment. For example, a system can include a heat exchanger, a control module, a primary fluid delivery line (“FDL”), an intravenous catheter, and a peristaltic pump. The control module can include at least a hydraulic system configured to provide a temperature-controlled fluid. The primary FDL can be configured to convey the temperature-controlled fluid to the heat exchanger as a supply fluid and back to the hydraulic system as a return fluid. The intravenous catheter can include a primary lumen configured to convey blood of the patient to the heat exchanger as well as a secondary lumen configured to convey the blood back to the patient using the peristaltic pump. The catheter can also include a thermistor for determining a core body temperature of the patient to ensure the patient is in a hyperthermic state before administering a cancer treatment to the patient.

### PRIORITY

This application claims the benefit of priority to U.S. Provisional Patent Application No. 63/158,263, filed Mar. 8, 2021, which is incorporated by reference in its entirety into this application.

### BACKGROUND

Hyperthermic treatment is the treatment of disease through local, regional, or whole-body heating. Whole-body heating is typically reserved for hyperthermic treatment of metastatic diseases including cancer. Such whole-body heating is often achieved by simply wrapping patients in blankets or heating patients' rooms; however, other more complex methods of whole-body heating have been used. A challenge in whole-body heating has been in determining and maintaining core temperatures during hyperthermic treatments. Therefore, systems and methods addressing this challenge are needed, particularly for the hyperthermic treatment of cancer, which is a leading cause of death year-over-year the world over.

Disclosed herein are systems and methods for hyperthermic cancer treatment.

### SUMMARY

Disclosed herein is a system for hyperthermic cancer treatment. The system is configured to induce hyperthermia in a patient for simultaneous cancer treatment. The system includes, in some embodiments, a heat exchanger, a control module, a primary fluid delivery line (“FDL”), an intravenous (“IV”) catheter, and a peristaltic pump. The control module includes at least a hydraulic system configured to provide a temperature-controlled system fluid. The primary FDL is configured to convey the temperature-controlled system fluid as a supply fluid to the heat exchanger. The primary FDL is also configured to convey a return fluid back to the hydraulic system. The IV catheter includes two-or-more lumens. A primary lumen of the two-or-more lumens is configured to convey blood of the patient to the heat exchanger. A secondary lumen of the two-or-more lumens is configured to convey the blood back to the patient. The peristaltic pump is configured to pump the blood from the patient to the heat exchanger. The peristaltic pump is also configured to pump the blood back to the patient.

In some embodiments, the two-or-more lumens include a tertiary lumen. The tertiary lumen is configured for IV administration of a solution of one or more chemotherapy agents or one or more immunotherapy agents to the patient.

In some embodiments, the catheter further includes a thermistor in a distal portion of the catheter. The thermistor is configured for determining a core temperature of the patient.

In some embodiments, the catheter further including a thermistor connector in a proximal portion of the catheter. The thermistor connector is a power-and-data connector configured for a wired connection to the control module or an intervening device between the thermistor connector and the control module.

In some embodiments, the heat exchanger includes a thermistor configured for determining a core body temperature of the patient.

In some embodiments, the heat exchanger and the peristaltic pump are in a heat-exchange module separate from the control module.

In some embodiments, the heat exchanger and the peristaltic pump are integrated into the control module.

In some embodiments, the hydraulic system further includes a heater configured for fluid heating, a chiller evaporator configured for fluid cooling, a hydraulic-system outlet, and a hydraulic-system inlet. The heater and the chiller evaporator, together, are configured to provide the temperature-controlled system fluid. The hydraulic-system outlet is configured for discharging the supply fluid from the hydraulic system. The hydraulic-system inlet is configured for charging the hydraulic system with the return fluid to continue to produce the temperature-controlled system fluid.

In some embodiments, the control module further includes one or more processors, primary memory, and instructions stored in the primary memory configured to instantiate one or more processes for hyperthermic cancer treatment with the control module. The one-or-more processes include a temperature-adjusting process. The temperature-adjusting process is configured to adjust a temperature of the temperature-controlled system fluid in accordance with core-temperature measurements to compensate for any deviance from a programmed temperature profile for the patient during the hyperthermic cancer treatment.

Also disclosed herein is another system for hyperthermic cancer treatment. The system is configured to induce hyperthermia in a patient for simultaneous cancer treatment. The system includes, in some embodiments, a control module, a primary FDL, one or more hydraulic pads, and a core temperature-determining means for determining a core temperature of the patient. The control module includes at least a hydraulic system. The hydraulic system is configured to provide a temperature-controlled system fluid. The primary FDL is configured to convey the temperature-controlled system fluid as a supply fluid from the hydraulic system. The primary FDL is also configured to convey a return fluid back to the hydraulic system. The one-or-more hydraulic pads are configured for placement on one or more portions of a body of the patient, respectively.

In some embodiments, the core temperature-determining means includes a tympanic thermometer, a rectal thermometer, a nasopharyngeal temperature probe, an esophageal temperature probe, a thermistor-tipped catheter, or a medical infrared thermometer for skin temperature adjusted with skin location and ambient temperature for core temperature.

In some embodiments, each pad of the one-or-more hydraulic pads includes a multilayered pad body, a pad inlet connector, and a pad outlet connector. The pad body includes a conduit layer and a thermally conductive adhesive layer over the conduit layer. The conduit layer includes one or more conduits. The one-or-more conduits are configured to convey the supply fluid from the hydraulic system. The one-or-more conduits are also configured to convey the return fluid back to the hydraulic system. The adhesive layer is configured for placement on a portion of the one-or-more portions of the body of the patient. The pad inlet connector includes a pad inlet. The pad inlet is configured for charging the conduit layer with the supply fluid. The pad outlet connector includes a pad outlet. The pad outlet is configured for discharging the return fluid from the conduit layer.

In some embodiments, the pad body further includes an impermeable film between the conduit layer and the adhesive layer. The impermeable film is configured to retain the supply fluid in the conduit layer.

In some embodiments, the adhesive layer includes a hydrogel. The hydrogel is selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, each pad of the one-or-more hydraulic pads further includes a release liner over the adhesive layer in a ready-to-use state of the pad. The release liner is configured to maintain integrity of at least the adhesive layer prior to use of the pad.

In some embodiments, the system further includes a secondary FDL for each pad of the one-or-more hydraulic pads. The secondary FDL is configured to convey the supply fluid from the primary FDL. The secondary FDL is also configured to convey the return fluid back to the primary FDL. The secondary FDL is split at a pad-connecting end of the secondary FDL. The pad-connecting end of the secondary FDL includes a pair of secondary FDL connectors. The pair of secondary FDL connectors includes a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

In some embodiments, the hydraulic system further includes a heater configured for fluid heating, a chiller evaporator configured for fluid cooling, a hydraulic-system outlet, and a hydraulic-system inlet. The heater and the chiller evaporator, together, are configured to provide the temperature-controlled system fluid. The hydraulic-system outlet is configured for discharging the supply fluid from the hydraulic system. The hydraulic-system inlet is configured for charging the hydraulic system with the return fluid to continue to produce the temperature-controlled system fluid.

In some embodiments, the control module further includes one or more processors, primary memory, and instructions stored in the primary memory configured to instantiate one or more processes for hyperthermic cancer treatment with the control module. The one-or-more processes including a temperature-adjusting process. The temperature-adjusting process is configured to adjust a temperature of the temperature-controlled system fluid in accordance with core-temperature measurements to compensate for any deviance from a programmed temperature profile for the patient during the hyperthermic cancer treatment.

Also disclosed herein is another system for hyperthermic cancer treatment. The system is configured to induce hyperthermia in a patient for simultaneous cancer treatment. The system includes, in some embodiments, a control module, a primary cable, one or more thermoelectric pads, and a core temperature-determining means for determining a core temperature of the patient. The control module includes one or more processors, primary memory, and instructions stored in the primary memory configured to instantiate one or more processes for operating a plurality of thermoelectric devices. The one-or-more thermoelectric pads are configured for placement on one or more portions of a body of the patient, respectively. Each pad of the one-or-more thermoelectric pads includes one or more thermoelectric devices. The one-or-more thermoelectric devices are operable by the control module by way of at least the primary cable.

In some embodiments, the core temperature-determining means includes a tympanic thermometer, a rectal thermometer, a nasopharyngeal temperature probe, an esophageal temperature probe, a thermistor-tipped catheter, or a medical infrared thermometer for skin temperature adjusted with skin location and ambient temperature for core temperature.

In some embodiments, each pad of the one-or-more thermoelectric pads includes a multilayered pad body and a pad connector. The pad body includes a thermoelectric layer and a thermally conductive adhesive layer over the thermoelectric layer. The thermoelectric layer includes the one-or-more thermoelectric devices. The one-or-more thermoelectric devices are configured to undergo a temperature change upon application of a voltage across the one-or-more thermoelectric devices. The adhesive layer is configured for placement on a portion of the one-or-more portions of the body of the patient. The pad connector is configured for establishing an operable connection with the control module.

In some embodiments, the adhesive layer includes a hydrogel. The hydrogel is selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, each pad of the one-or-more thermoelectric pads further includes a release liner over the adhesive layer in a ready-to-use state of the pad. The release liner is configured to maintain integrity of at least the adhesive layer prior to use of the pad.

In some embodiments, the one-or-more processes include a temperature-adjusting process. The temperature-adjusting process is configured to adjust a temperature of the plurality of thermoelectric devices in accordance with core-temperature measurements to compensate for any deviance from a programmed temperature profile for the patient during the hyperthermic cancer treatment.

Also disclosed herein is a method for hyperthermic cancer treatment. The method includes, in some embodiments, a hyperthermia-inducing step, a deploying step, and a cancer treatment-administering step. The hyperthermia-inducing step includes inducing hyperthermia in a patient with a system for hyperthermic cancer treatment. The deploying step includes deploying a core temperature-determining means for determining a core temperature of the patient. The deploying step enables a control module of the system to adjust the core temperature of the patient to compensate for any deviance from a programmed temperature profile for the patient during the hyperthermic cancer treatment. The cancer treatment-administering step includes administering a cancer treatment including radiation therapy, chemotherapy, or immunotherapy while the patient is in a hyperthermic state, thereby complementing the cancer treatment.

In some embodiments, the deploying step includes inserting an IV catheter into the patient. The catheter includes a thermistor in a distal portion of the catheter configured for determining a core temperature of the patient.

In some embodiments, the method further includes a thermistor-connecting step. The thermistor-connecting step includes connecting a thermistor connector of the catheter to the control module or an intervening device between the thermistor connector and the control module. The thermistor-connecting step powers the thermistor and enables signals or data corresponding to microammeter-measured currents from a microammeter in series with the thermistor to be communicated to the control module.

In some embodiments, the hyperthermia-inducing step includes conveying blood of the patient to a heat exchanger of the system by way of a primary lumen of the catheter. The hyperthermia-inducing step also includes conveying the blood back to the patient by way of a secondary lumen of the catheter. The heat exchanger is configured to exchange heat between the blood and a temperature-controlled system fluid conveyed to the heat exchanger.

In some embodiments, the cancer treatment-administering step includes intravenously administering a solution of one or more chemotherapy agents to the patient by way of a tertiary lumen of the catheter.

In some embodiments, the cancer treatment-administering step includes intravenously administering a solution of one or more immunotherapy agents to the patient by way of a tertiary lumen of the catheter.

In some embodiments, the method further includes a pad-placing step. The pad-placing step includes placing a pad on a portion of a body of the patient with a thermally conductive adhesive layer of a multilayered pad body of the pad in contact with skin of the portion of the patient's body.

In some embodiments, the hyperthermia-inducing step includes charging a conduit layer of the pad with a supply fluid of a temperature-controlled system fluid to exchange heat between the supply fluid and the portion of the body of the patient by thermal conduction through the adhesive layer. The supply fluid is provided by a hydraulic system of the control module by way of a combination of fluidly connected FDLs. The FDLs include a secondary FDL and a primary FDL.

In some embodiments, the hyperthermia-inducing step includes applying a voltage across one or more thermoelectric devices of a thermoelectric layer of the pad to exchange heat between the one-or-more thermoelectric devices and the portion of the body of the patient by thermal conduction through the adhesive layer. The voltage is applied by the control module by way of a primary cable between the pad and the control module.

In some embodiments, the method further includes a release liner-removal step. The release liner-removal step includes removing a release liner of the pad to reveal the adhesive layer before the pad-placing step. The release liner is configured to maintain integrity of at least the adhesive layer prior to using the pad.

In some embodiments, the deploying step includes deploying a tympanic thermometer, a rectal thermometer, a nasopharyngeal temperature probe, an esophageal temperature probe, a thermistor-tipped catheter, or a medical infrared thermometer for skin temperature adjusted with skin location and ambient temperature for core temperature.

In some embodiments, the cancer treatment-administering step includes intravenously administering a solution of one or more chemotherapy agents to the patient by way of the catheter.

In some embodiments, the cancer treatment-administering step includes intravenously administering a solution of one or more immunotherapy agents to the patient by way of the catheter.

In some embodiments, the method further includes a normothermia-inducing step. The normothermia-inducing step includes inducing normothermia in the patient after the cancer treatment-administering step.

These and other features of the concepts provided herein will become more apparent to those of skill in the art in view of the accompanying drawings and following description, which describe particular embodiments of such concepts in greater detail.

### DRAWINGS

FIG. 1 illustrates a system for hyperthermic cancer treatment in accordance with some embodiments.

FIG. 2 illustrates another system for hyperthermic cancer treatment in accordance with some embodiments.

FIG. 3 illustrates a hydraulic system of a control module in accordance with some embodiments.

FIG. 4A illustrates left and right hydraulic pads for a torso of a patient in accordance with some embodiments.

FIG. 4B illustrates left and right hydraulic pads for legs of a patient in accordance with some embodiments.

FIG. 5 illustrates a multilayered pad body of a hydraulic pad in accordance with some embodiments.

FIG. 6 illustrates yet another system for hyperthermic cancer treatment in accordance with some embodiments.

FIG. 7 illustrates a multilayered pad body of a thermoelectric pad in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, hyperthermic treatment is the treatment of disease through local, regional, or whole-body heating. Whole-body heating is typically reserved for hyperthermic treatment of metastatic diseases including cancer. Such whole-body heating is often achieved by simply wrapping patients in blankets or heating patients' rooms; however, other more complex methods of whole-body heating have been used. A challenge in whole-body heating has been in determining and maintaining core temperatures during hyperthermic treatments. Therefore, systems and methods addressing this challenge are needed, particularly for the hyperthermic treatment of cancer, which is a leading cause of death year-over-year the world over.

Disclosed herein are systems and methods for hyperthermic cancer treatment. However, it should be understood that patients with diseases other than cancer can benefit from treatments of those diseases while in hyperthermic states. For example, systems and methods disclosed herein can be adapted to induce hyperthermia in patients for IV administration of a solution of one or more antibiotic agents to treat bacterial diseases such as Lyme disease. In another example, systems and methods disclosed herein can be adapted to induce hyperthermia in patients for IV administration of a solution of one or more antiviral agents to treat viral diseases.

### Systems for Hyperthermic Cancer Treatment

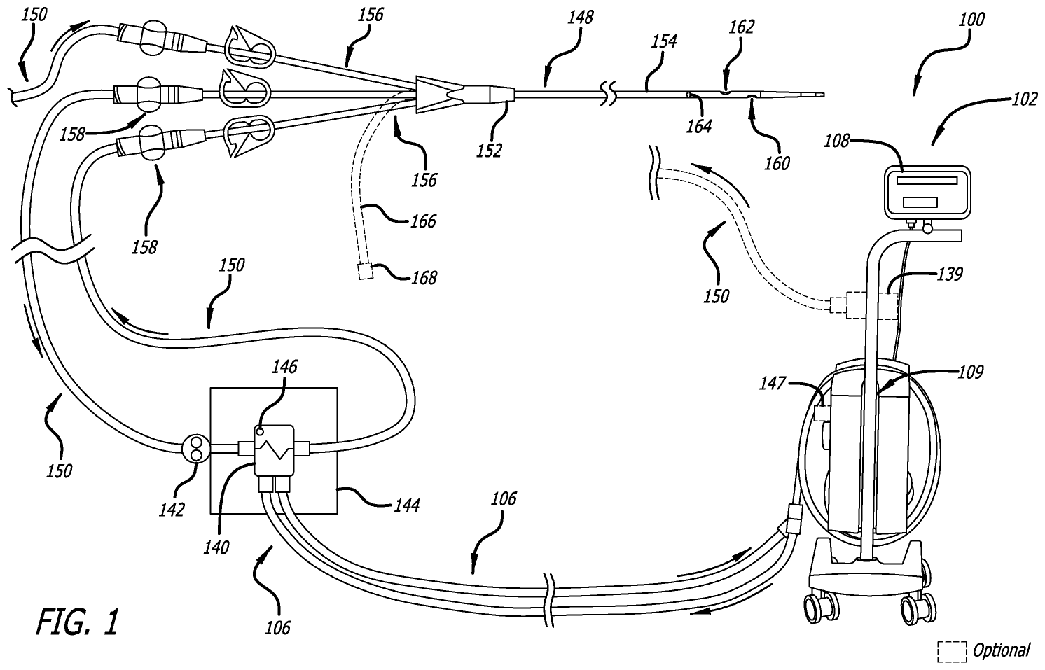

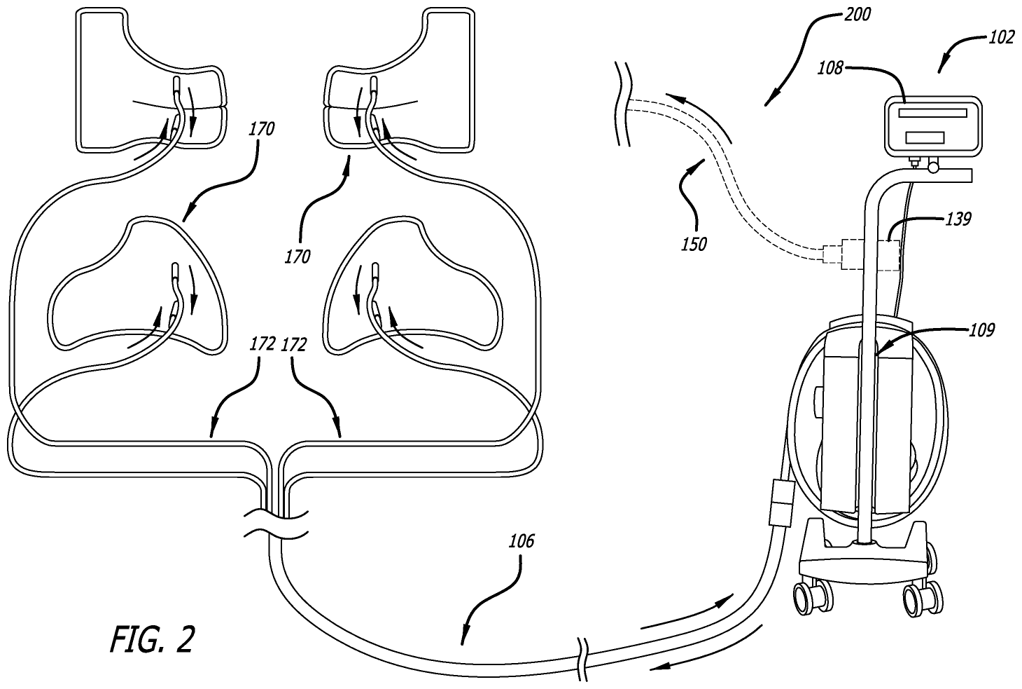

FIGS. 1 and 2 illustrate systems 100 and 200 for hyperthermic cancer treatment in accordance with some embodiments.

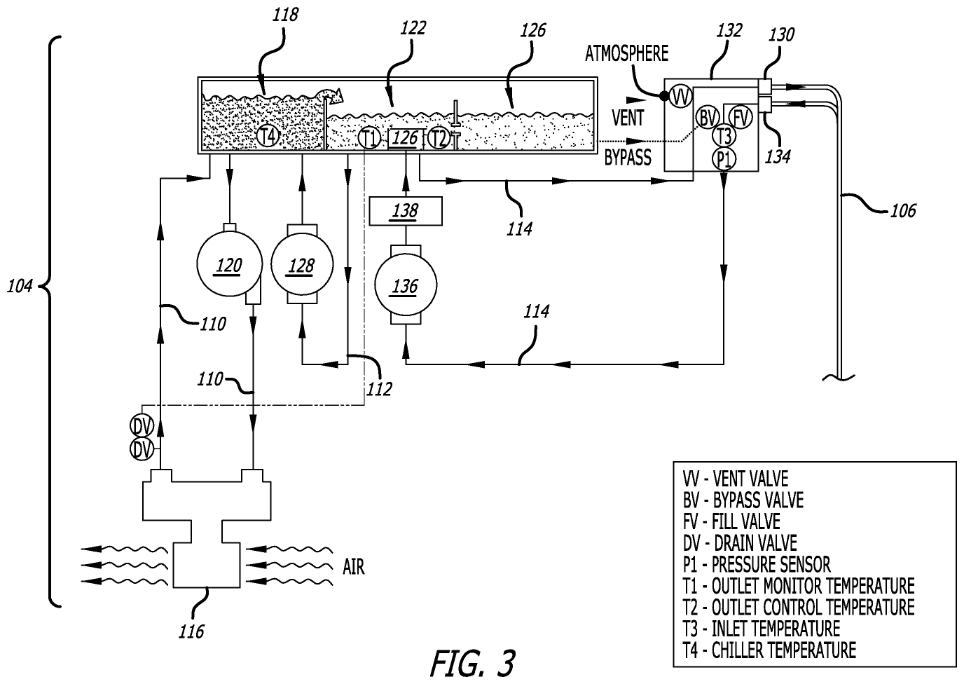

As shown, each system of the systems 100 and 200 can include a control module 102 including a hydraulic system 104 (see FIG. 3) configured to provide a temperature-controlled system fluid for inducing hyperthermia in a patient for cancer treatment or bringing the patient back to a state of normothermia. At least one primary FDL 106 of the system 100 or 200 can be configured to convey the temperature-controlled system fluid as a supply fluid to one or more downstream devices (e.g., the heat exchanger 140 or the one-or-more hydraulic pads 170 set forth below) as well as convey a return fluid back to the hydraulic system 104. Each system of the systems 100 and 200 can further include a core temperature-determining means (e.g., a thermistor-equipped heat exchanger such as the heat exchanger 140 with the thermistor 146, a thermistor-tipped catheter such as the catheter 148, a tympanic thermometer, a rectal thermometer, a thermistor-tipped indwelling urinary catheter, a nasopharyngeal temperature probe, an esophageal temperature probe, a medical infrared thermometer for skin temperature adjusted with skin location and ambient temperature for core temperature, etc.) directly or indirectly communicatively coupled to the control module 102 like the catheter 148 set forth herein for determining a core temperature of the patient while connected to the system 100 or 200. Each system of the systems 100 and 200 differs from the other system in the one-or-more downstream devices used to induce hyperthermia in the patient. As such, description of features common to each system of the systems 100 and 200 is set forth immediately below followed by description of features primarily found in the system 100 and, subsequently, in the system 200.

The control module 102 can include a console 108 with an integrated display screen configured as a touchscreen for operating the control module 102. The console 108 can include one or more processors, primary and secondary memory, and instructions stored in the primary memory configured to instantiate one or more processes for hyperthermic cancer treatment with the control module 102. For example, the one-or-more processes can include a temperature-adjusting process. The temperature-adjusting process is configured to adjust a temperature of the temperature-controlled system fluid in accordance with core-temperature measurements for a patient to compensate for any deviance from a programmed temperature profile for the patient during hyperthermic cancer treatment whether attaining a hyperthermic state (e.g., 99.0-104.0° F. such as 101.0-103.0° F. or an intervening temperature thereof in tenths of a degree), maintaining the hyperthermic state, or returning the patient to a normothermic state.

The control module 102 can include a channel 109 configured to accept therein a pole of an IV-pole cart, IV-pole stand, or the like such that the control module 102 can be conveniently mounted thereon.

FIG. 3 illustrates the hydraulic system 104 of the control module 102 in accordance with some embodiments.

The hydraulic system 104 can include a chiller circuit 110, a mixing circuit 112, and a circulating circuit 114 for providing the temperature-controlled system fluid.

The chiller circuit 110 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 122 set forth below to produce the supply fluid with an appropriate temperature for inducing or maintaining hyperthermia in a patient or bringing the patient back to a state of normothermia. The chiller circuit 110 can include a chiller evaporator 116 configured for the cooling of the system fluid passing therethrough. The system fluid for the cooling by the chiller evaporator 116 is provided by a chiller tank 118 using a chiller pump 120 of the chiller circuit 110.

The mixing circuit 112 can be configured for mixing spillover of the cooled fluid from the chiller tank 118 with a mixed fluid in a mixing tank 122 of the mixing circuit 112. The mixing circuit 112 can include a heater 124 in the mixing tank 122 configured for heating the mixed fluid to produce a heated fluid, which heated fluid, in turn, can be for mixing with the cooled fluid in the mixing tank 122 to produce the supply fluid with the appropriate temperature for inducing or maintaining hyperthermia in a patient or bringing the patient back to a state of normothermia. The mixing circuit 112 can also include an overflow tank 126 and a mixing pump 128. The mixing pump 128 can be configured to pump the system fluid from the mixing tank 122 into the chiller tank 118 for producing the cooled fluid as well as the spillover of the cooled fluid for the mixing tank 122.

The circulating circuit 114 can be configured for circulating the system fluid for inducing or maintaining hyperthermia in a patient or bringing the patient back to a state of normothermia when the system fluid is of an appropriate temperature therefor. Such a configuration can include an outlet 130 of a manifold 132 configured to discharge the supply fluid from the hydraulic system 104 to the one-or-more downstream devices. Such a configuration can also include an inlet 134 of the manifold 132 configured to charge the hydraulic system 104 with the return fluid from the one-or-more downstream devices for continuing to produce the supply fluid. The circulating circuit 114 can include a circulation pump 136 directly or indirectly governed by a flow meter 138 of the circulating circuit 114 for circulating the system fluid.

The system 100 or 200 can further include an infusion pump 139. The infusion pump 139 can be configured for IV administration of a solution of one or more chemotherapy agents or one or more immunotherapy agents (e.g., immunomodulators) to the patient by way of a secondary FDL of the one-or-more secondary FDLs 150 set forth below. The infusion pump 139 can be configured to continuously administer as little as 0.1 mL of the solution per hour to the patient, periodically administer boluses of the solution to the patient, or a combination thereof (e.g., continuously administer a particular volume of the solution to the patient over time with additional, periodic boluses of the solution to the patient over the same time).

Adverting to FIG. 1, the system 100 can further include a heat exchanger 140 and a peristaltic pump 142, as well as the catheter 148 and the one-or-more secondary FDLs 150 set forth below.

The heat exchanger 140 and the peristaltic pump 142 can be integrated into the control module 102, or the heat exchanger 140 and the peristaltic pump 142 can be integrated into a heat-exchange module 144 separate from the control module 102. The heat exchanger 140 and the peristaltic pump 142 being integrated into the control module 102 provides one convenient unit (i.e., the control module 102) for hyperthermic cancer treatment. However, the heat exchanger 140 and the peristaltic pump 142 being integrated into the separate heat-exchange module 144 allows the heat-exchange module 144 and, thus, the peristaltic pump 142 to be placed closer to the patient, thereby reducing line lengths for the one-or-more secondary FDLs 150 and inherent pulsation therein due to the pumping mechanism of the peristaltic pump 142.

The heat exchanger 140 can be configured to exchange heat between the supply fluid provided by the hydraulic system 104 and blood of a patient provided by the catheter 148 by way of the one-or-more secondary FDLs 150. The heat exchanger 140 can be a shell-and-tube heat exchanger configured for parallel flow, counter flow, or cross-flow. However, it should be understood the heat exchanger 140 is not limited to the shell-and-tube heat exchanger. Indeed, the heat exchanger 140 can be any heat exchanger of a number of other heat exchangers provided the blood of the patient is contained in a closed, single-use fluidly connected system.

The peristaltic pump 142 can be configured to pump the blood from the patient to the heat exchanger 140 as well as pump the blood back to the patient by way of the one-or-more secondary FDLs 150. Because the peristaltic pump 142 is multi-use equipment as opposed to single-use, disposable equipment like the one-or-more secondary FDLs 150, the peristaltic pump 142 can be configured to easily switch out the one-or-more secondary FDLs 150 for different patients.

The system 100 can further include a thermistor 146 or a pair of thermistors disposed in the heat exchanger 140 configured for determining a core temperature of a patient when the catheter 148 is intravenously disposed in the patient. For example, the thermistor 146 and a microammeter in series with the thermistor 146 can be disposed in the heat exchanger 140 at or near a blood inlet of the heat exchanger 140 for determining the core temperature of the patient by way of influent blood. If the pair of thermistors is present, one thermistor of the pair of thermistors can be disposed at or near the blood inlet of the heat exchanger 140 for determining the core temperature of the patient by way of the influent blood, and the other thermistor of the pair of thermistors can be disposed at the blood outlet of the heat exchanger 140 for determining the temperature of the effluent blood. Being that each thermistor of the pair of thermistors is in series with a microammeter, each microammeter of a pair of microammeters is likewise respectively disposed at or near the blood inlet and the blood outlet of the heat exchanger 140. Advantageously for the pair of thermistors, the temperature of the influent blood and the temperature of the effluent blood can be used to determine a difference in temperature ΔT for an instant indication of how far the patient is from achieving a desired hyperthermic state. In addition, the temperature-adjusting process of the control module 102 set forth above can utilize the difference in temperature ΔT over time (ΔT/t) in accordance with, for example, Newtons' Law of Heating to determine when the patient will achieve the desired hypothermic state. The temperature of the temperature-controlled system fluid can be subsequently increased or decreased as needed to hasten or delay the patient in achieving a desired hyperthermic state.

The system 100 can alternatively or additionally include a control module-side thermistor connector 147 configured for a direct or indirect wired connection to the catheter-side thermistor connector 168 if the thermistor 164 is present in the catheter 148. The thermistor connector 147 can be configured to power the thermistor 164 of the catheter 148 or communicate with the thermistor 164 (e.g., receive signals or data corresponding to measured currents for the thermistor 164). Alternatively, the control module 102 includes a wireless module configured for a wireless connection to an intervening wireless device between the control module 102 and the catheter 148 to communicate with the thermistor 164. In such embodiments, the intervening wireless device can be configured to power the thermistor of the catheter 148.

Adverting to FIG. 2, the system 200 can further include the one-or-more hydraulic pads 170 and the one-or-more secondary FDLs 172 set forth below.

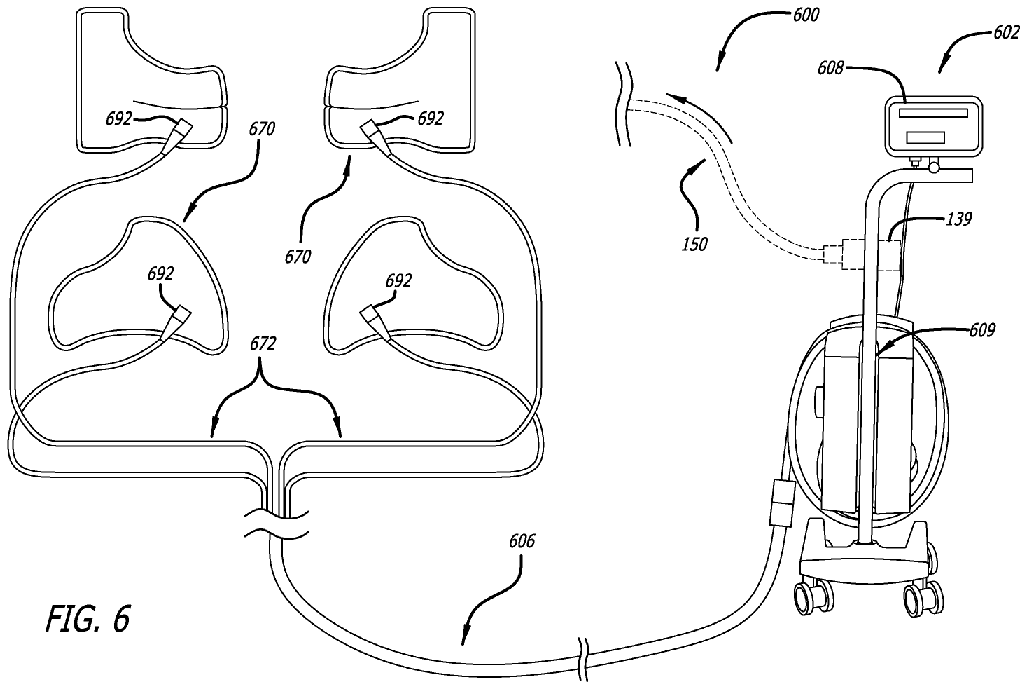

FIG. 6 illustrates a system 600 for hyperthermic cancer treatment in accordance with some embodiments.

As shown, the system 600 can include a control module 602 configured for inducing hyperthermia in a patient for cancer treatment or bringing the patient back to a state of normothermia. A primary cable 606 of the system 600, optionally with the one-or-more secondary cables 672 set forth below, can be configured to power or control one or more downstream devices (e.g., the one-or-more thermoelectric pads 670 set forth below). The system 600 can further include a core temperature-determining means (e.g., a thermistor-equipped heat exchanger such as the heat exchanger 140 with the thermistor 146, a thermistor-tipped catheter such as the catheter 148, a tympanic thermometer, a rectal thermometer, a thermistor-tipped indwelling urinary catheter, a nasopharyngeal temperature probe, an esophageal temperature probe, a medical infrared thermometer for skin temperature adjusted with skin location and ambient temperature for core temperature, etc.) directly or indirectly communicatively coupled to the control module 602 like the catheter 148 set forth herein for determining a core temperature of the patient while connected to the system 600.

Like the control module 102, the control module 602 can include a console 608 with an integrated display screen configured as a touchscreen for operating the control module 602. The console 608 can include one or more processors, primary and secondary memory, and instructions stored in the primary memory configured to instantiate one or more processes for hyperthermic cancer treatment with the control module 602 including operating a plurality of thermoelectric devices such as those of the one-or-more thermoelectric pads 670 set forth below. The one-or-more processes can also include a temperature-adjusting process. The temperature-adjusting process is configured to adjust a temperature of the plurality of thermoelectric devices in accordance with core-temperature measurements for a patient to compensate for any deviance from a programmed temperature profile for the patient during hyperthermic cancer treatment whether attaining a hyperthermic state (e.g., 99.0-104.0° F. such as 101.0-103.0° F. or an intervening temperature thereof in tenths of a degree), maintaining the hyperthermic, or returning the patient to a normothermic state.

Also like the control module 102, the control module 602 can include a channel 609 configured to accept therein a pole of an IV-pole cart, IV-pole stand, or the like such that the control module 602 can be conveniently mounted thereon.

The system 600 can further include the infusion pump 139. As set forth above for the systems 100 and 200, the infusion pump 139 can be configured for IV administration of the solution of the one-or-more chemotherapy agents or the one-or-more immunotherapy agents (e.g., immunomodulators) to the patient by way of a secondary FDL of the one-or-more secondary FDLs 150 set forth above. Again, the infusion pump 139 can be configured to continuously administer as little as 0.1 mL of the solution per hour to the patient, periodically administer boluses of the solution to the patient, or a combination thereof (e.g., continuously administer a particular volume of the solution to the patient over time with additional, periodic boluses of the solution to the patient over the same time).

The system 600 can further include the one-or-more thermoelectric pads 670 set forth below.

It should be understood that while the catheter 148 and the one-or-more secondary FDLs 150 set forth below are single-use, disposable devices as opposed to multiuse, capital equipment, they can be considered part of the system 100 set forth above for the purpose this disclosure. The one-or-more hydraulic pads 170 and the one-or-more secondary FDLs 172 set forth below are also single-use, disposable devices; they can similarly be considered part of the system 200 set forth above for the purpose of this disclosure. Likewise, the one-or-more thermoelectric pads 670 set forth below are single-use, disposable devices but can be considered part of the system 600 set forth above for the purpose this disclosure.

### Catheters for Hyperthermic Cancer Treatment

FIG. 1 illustrates the system 100 for hyperthermic cancer treatment including an IV catheter 148 and one or more secondary FDLs 150 in accordance with some embodiments.

As shown, the catheter 148 can include a hub 152, a catheter tube 154 distally extending from the hub 152, and a number of extension legs 156 proximally extending from the hub 152. Being that the catheter 148 is configured to convey at least blood of a patient as set forth below, the catheter 148 can be a single-use, disposable catheter.

The hub 152 can be coupled to a proximal portion of the catheter tube 154 such as by insertion of the proximal portion of the catheter tube 154 into a bore in a distal portion of the hub 152. While not shown, the hub 152 can also include a number of bores in a proximal portion of the hub 152 corresponding in number to the number of extension legs 156. The number of bores in the distal portion of the hub 152 can be configured to accept insertion of the number of extension legs 156 into the number of bores.

The hub 152 can be furcated in accordance with a number of lumens extending through the catheter 148. For example, the hub 152 can be bifurcated for a diluminal catheter or trifurcated for a triluminal catheter. Depending upon a chosen method of manufacturing, the hub 152 can be molded over a number of core pins for a number of fluid pathways longitudinally extending through the hub 152 configured to fluidly connect a number of catheter-tube lumens of the catheter tube 154 to a number of extension-leg lumens of the number of extension legs 156. Alternatively, the hub 152 can be molded over a number of cannulas longitudinally extending through the hub 152 configured to fluidly connect the number of catheter-tube lumens of the catheter tube 154 to the number of extension-leg lumens of the number of extension legs 156.

The number of extension legs 156 can extend from the hub 152 by way of their distal portions. The number of extension legs 156 can be equal to the number of lumens extending through the catheter 148. For example: If the catheter 148 is a diluminal catheter, two extension legs can extend from the hub 152. If the catheter 148 is a triluminal catheter, three extension legs can extend from the hub 152. If the catheter 148 is a tetraluminal catheter, four extension legs can extend from the hub 152.

The catheter 148 can also include a number of Luer connectors 158 for fluidly connecting the one-or-more secondary FDLs 172 set forth below to the catheter 148. Each extension leg of the number of extension legs 156 can include a Luer connector of the number of Luer connectors 158 coupled to a proximal portion of the extension leg. Optionally, as set forth below, one Luer connector of the number of Luer connectors 158 is instead the thermistor connector 168. Given the foregoing, the number of Luer connectors 158 including the optional thermistor connector 168 can be equal to the number of extension legs 156, which number of extension legs 156, in turn, can be equal to the number of lumens extending through the catheter 148. For example: If the catheter 148 is a diluminal catheter, two extension legs can extend from the hub 152 and two Luer connectors can be respectively coupled to the two extension legs. If the catheter 148 is a triluminal catheter, three extension legs can extend from the hub 152 and three Luer connectors can be respectively coupled to the three extension legs. If the catheter 148 is a tetraluminal catheter, four extension legs can extend from the hub 152 and four Luer connectors can be respectively coupled to the three extension legs.

The catheter 148 can be a multiluminal catheter such as the diluminal catheter or triluminal catheter set forth above or a tetraluminal catheter, a pentaluminal catheter, a hexaluminal catheter, or the like. When the catheter 148 is configured as a triluminal catheter as shown, the catheter 148 can include a primary lumen, a secondary lumen, and a tertiary lumen. The primary lumen, which may be considered a distal lumen, can extend from an opening in a proximal end of a first Luer connector of the number of Luer connectors 158 to an opening in a tip or distal end of the catheter tube 154. The secondary lumen, which may be considered a medial lumen, can extend from an opening in a proximal end of a second Luer connector of the number of Luer connectors 158 to an eyelet 160 in a distal portion of the catheter tube 154. The tertiary lumen, which may be considered a proximal lumen, can extend from an opening in a proximal end of a third Luer connector of the number of Luer connectors 158 to an eyelet 162 proximal of the eyelet 160 in the distal portion of the catheter tube 154.

The primary lumen of the catheter 148 when a multiluminal catheter such as a diluminal or triluminal catheter can be configured to convey blood from a patient to the heat exchanger 140. The secondary lumen of such a catheter can be configured to convey the blood from the heat exchanger 140 back to the patient. For the diluminal configuration of the catheter 148, IV administration of the solution of the one-or-more chemotherapy agents or the one-or-more immunotherapy agents (e.g., immunomodulators) to the patient must be given by way of another IV device; however, for the triluminal configuration of the catheter 148, the tertiary lumen can be configured for the IV administration of the solution of the one-or-more chemotherapy agents or the one-or-more immunotherapy agents to the patient, which is advantageous in that only one IV device is needed. Notwithstanding the foregoing, it should be understood the lumens of the catheter 148 can be configured such that a clinician can choose which lumens to use for conveying the blood of the patient to and from the heat exchanger 140 as well as the lumen to use for administering the solution of the one-or-more chemotherapy agents or the one-or-more immunotherapy agents to the patient.

The catheter 148 can also include a thermistor 164 in a distal portion of the catheter 148. For example, the thermistor 164 can be disposed in a wall of the catheter tube 154. The thermistor 164 can be configured for determining a core temperature of a patient when the catheter 148 is intravenously disposed in the patient. Electrical leads distally extending from the hub 152 to the thermistor 164 along with a microammeter in series with the thermistor 164 can also be disposed in the wall of the catheter tube 154; however, the electrical leads and the microammeter can alternatively be disposed in a lumen of the catheter 148 dedicated thereto. Regardless, a remainder of the electrical leads proximally extending from the hub 152 can be disposed in an extension lead 166 or an extension leg of the number of extension legs 156 terminating with a thermistor connector 168 in a proximal portion of the catheter 148.

The thermistor connector 168 can be a power-and-data connector configured for a wired connection to the control module 102 or an intervening device (e.g., a dedicated device, a smartphone including a dedicated software application, etc.) between the thermistor connector 168 and the control module 102. For example, the thermistor connector 168 can be configured to directly connect to the control module 102 or indirectly connect to the control module 102 through an intervening cable for powering the thermistor 164 and communicating signals corresponding to microammeter-measured currents from the microammeter. Alternatively, the thermistor connector 168 can be configured to directly connect to the intervening device or indirectly connect to the intervening device through an intervening cable for powering the thermistor 164 and wirelessly communicating signals corresponding to microammeter-measured currents from the microammeter to the control module 102 by way of the intervening device.

The one-or-more secondary FDLs 150 can be configured to convey blood of a patient or the solution of the one-or-more chemotherapy agents or the one-or-more immunotherapy agents (e.g., immunomodulators) for the patient. As shown, a secondary FDL of the one-or-more FDLs 150 can be configured to convey the blood of the patient from the catheter 148 (e.g., from the primary lumen of the catheter 148) to the heat exchanger 140. A same or different secondary FDL of the one-or-more secondary FDLs 150 can be configured to convey the blood of the patient from the heat exchanger 140 to the catheter 148 (e.g., to the medial lumen of the catheter 148). Another FDL of the one-or-more FDLs 150 can be configured to convey the solution of the one-or-more chemotherapy agents or the one-or-more immunotherapy agents from the infusion pump 139, if present, or an IV infusion bag (not shown) to the patient.

### Pads for Hyperthermic Cancer Treatment

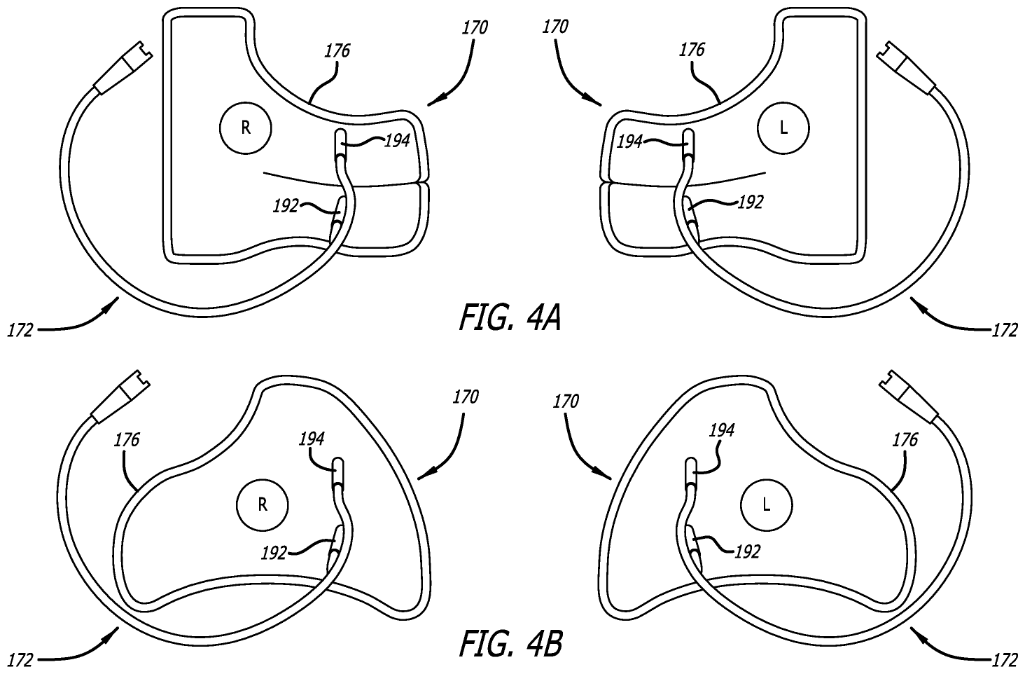

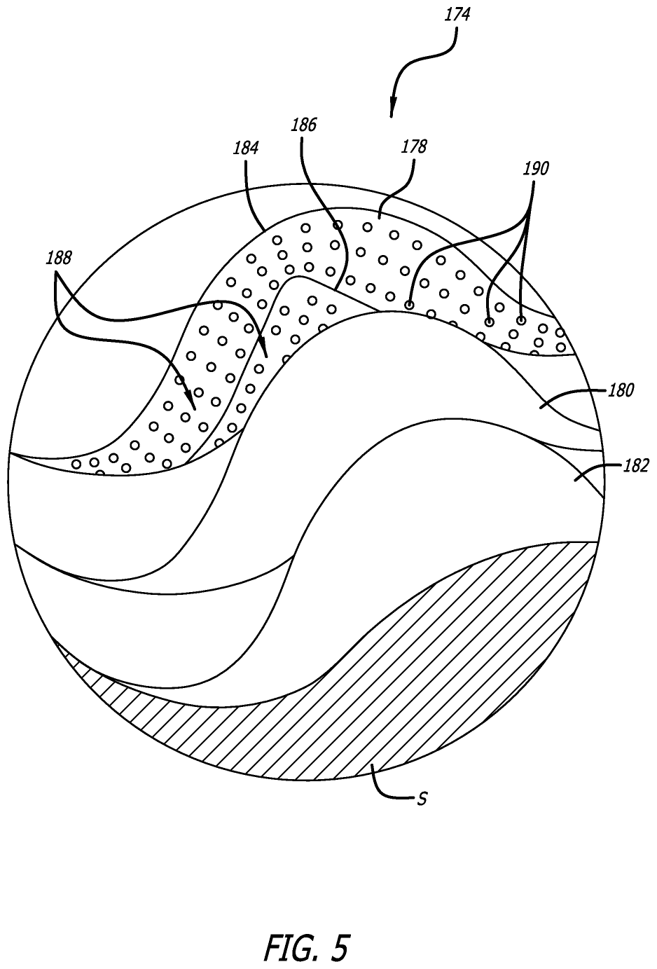

FIG. 1 illustrates the system 200 for hyperthermic cancer treatment including one or more hydraulic pads 170 and one or more secondary FDLs 172 in accordance with some embodiments. FIGS. 4A and 4B illustrate left and right pads of the one-or-more hydraulic pads 170 respectively for a torso and legs of a patient in accordance with some embodiments. However, the one-or-more hydraulic pads 170 can be configured for placement on any one or more respective portions of a body of such a patient, not just the torso or legs. FIG. 5 illustrates a multilayered pad body 174 of a pad of the one-or-more hydraulic pads 170 in accordance with some embodiments.

A pad of the one-or-more hydraulic pads 170 can include the pad body 174, a release liner 176 over the pad body 174, a pad inlet connector (not shown), and a pad outlet connector (shown). Such a pad can be configured for regional heating such as regional heating of a portion of a body of a patient for radiation therapy over that portion of the patient. Alternatively, a plurality of the one-or-more hydraulic pads 170 can be configured for whole-body heating for IV administration of the solution of one or more chemotherapy agents or the one-or-more immunotherapy agents to the patient.

The pad body 174 can include a conduit layer 178, an impermeable film 180 over the conduit layer 178, and a thermally conductive adhesive layer 182 over the impermeable film 180 and the conduit layer 178.

The conduit layer 178 can include a perimetrical wall 184 and one or more inner walls 186 extending from the conduit layer 178 toward the impermeable film 180. Together with the impermeable film 180, the perimetrical wall 184 and the one-or-more inner walls 186 of the conduit layer 178 form one or more conduits 188 configured to convey the supply fluid from the hydraulic system 104 through the conduit layer 178 as well as convey the return fluid back to the hydraulic system 104. A plurality of protrusions 190 extending from the conduit layer 178 toward the impermeable film 180 in the one-or-more conduits 188 can be configured to promote even flow of the supply fluid when the supply fluid is conveyed through the conduit layer 178. Such a conduit layer can be of a unitary piece of an insulating polymer (e.g., foam).

The impermeable film 180 can be configured to retain the supply fluid in the conduit layer 178 when the supply fluid is conveyed through the conduit layer 178. In addition, the impermeable film 180 can be configured to allow efficient energy transfer between the conduit layer 178 and the adhesive layer 182. Should the impermeable film 180 be breached by a sharp object like a needle, the impermeable film 180 can be configured to self-seal like a rubber septum.

The adhesive layer 182 can be configured for placement on skin S (see FIG. 5) of a portion (e.g., torso, leg, etc.) of a body of a patient for direct thermal conduction through the adhesive layer 182. While the adhesive layer 182 can be configured to conformably adhere to the patient for better thermal conduction, adherence of the adhesive layer 182 to the patient can be optimized to avoid irritating or wounding the patient upon removal of the pad.

The adhesive layer 182 can include a hydrogel or a silicone with optional additives to enhance thermal conductivity. The hydrogel can be selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

The release liner 176 can be over the adhesive layer 182 in a ready-to-use state of the pad. The release liner 176 can be configured to maintain integrity of at least the adhesive layer 182 prior to use of the pad.

The pad inlet connector and the pad outlet connector can be respectively inferred from the secondary FDL outlet connector 192 and the secondary FDL inlet connector 194 thereover in FIGS. 4A and 4B. The pad inlet connector can include a pad inlet configured for charging the conduit layer 178 with the supply fluid. The pad outlet connector can include a pad outlet configured for discharging the return fluid from the conduit layer 178.

Each secondary FDL of the one-or-more secondary FDLs 172 can be fluidly pre-connected to a pad of the one-or-more hydraulic pads 170 as sold. Indeed, a secondary FDL can be split at a pad-connecting end of the secondary FDL, which pad-connecting end of the secondary FDL can include a pair of secondary FDL connectors configured to connect to a pad of the one-or-more hydraulic pads 170. A secondary FDL inlet connector 192 of the pair of secondary FDL connectors can be fluidly pre-connected to the pad outlet connector of the pad as sold. A secondary FDL outlet connector 194 of the pair of secondary FDL connectors can be fluidly pre-connected to the inlet connector of the pad as sold. That said, the one-or-more secondary FDLs 172 need not be fluidly pre-connected to the one-or-more hydraulic pads 170 as sold. Notwithstanding the foregoing, once fluidly connected to a remainder of the system 200, each secondary FDL of the one-or-more secondary FDLs 172 can convey in a supply lumen thereof the supply fluid from the hydraulic system 104 by way of the primary FDL 106. Likewise, each secondary FDL of the one-or-more secondary FDLs 172 can convey in a return lumen thereof the return fluid from the hydraulic system 104 by way of the primary FDL 106.

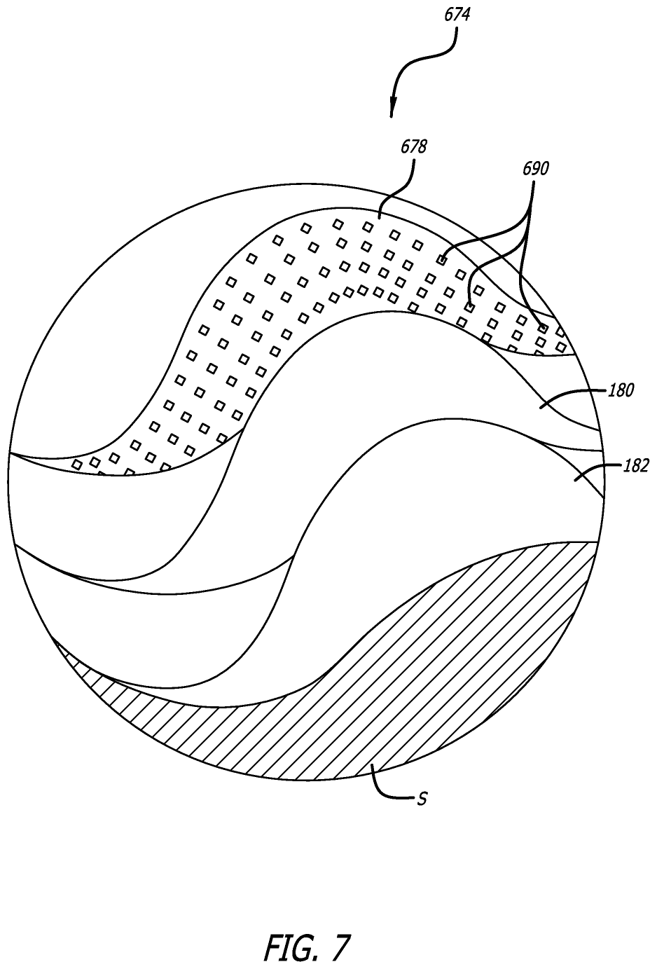

FIG. 6 illustrates the system 600 for hyperthermic cancer treatment including one or more thermoelectric pads 670 and one or more secondary cables 672 in accordance with some embodiments. While FIGS. 4A and 4B illustrate left and right pads of the one-or-more hydraulic pads 170 respectively for a torso and legs of a patient, the one-or-more thermoelectric pads 670 can be likewise be respectively configured for the torso and legs of a patient as shown in FIG. 6. However, the one-or-more thermoelectric pads 670 can be configured for placement on any one or more respective portions of a body of such a patient, not just the torso or legs. FIG. 7 illustrates a multilayered pad body 674 of a pad of the one-or-more thermoelectric pads 670 in accordance with some embodiments.

Like a pad of the one-or-more hydraulic pads 170, a pad of the one-or-more thermoelectric pads 670 can include the pad body 674, the release liner 176 over the pad body 674, and a pad connector (not shown). Such a pad can be configured for regional heating such as regional heating of a portion of a body of a patient for radiation therapy over that portion of the patient. Alternatively, a plurality of the one-or-more thermoelectric pads 670 can be configured for whole-body heating for IV administration of the solution of one or more chemotherapy agents or the one-or-more immunotherapy agents to the patient.

The pad body 674 can include a thermoelectric layer 678, the impermeable film 180 over the thermoelectric layer 678, and the thermally conductive adhesive layer 182 over the impermeable film 180 as well as the thermoelectric layer 678.

The thermoelectric layer 678 can include one-or-more thermoelectric devices 690 disposed in the thermoelectric layer 678. The one-or-more thermoelectric devices 690 are configured to undergo a temperature change upon application of a voltage across the one-or-more thermoelectric devices 690.

The impermeable film 180 can be configured to separate and electrically insulate the thermoelectric layer 678 from the adhesive layer 182. In addition, the impermeable film 180 can be configured to allow efficient energy transfer between the thermoelectric layer 678 and the adhesive layer 182.

The adhesive layer 182 can be configured for placement on skin S (see FIG. 7) of a portion (e.g., torso, leg, etc.) of a body of a patient for direct thermal conduction through the adhesive layer 182 as set forth above.

The release liner 176 can be over the adhesive layer 182 in a ready-to-use state of the pad as set forth above. The release liner 176 can be configured to maintain integrity of at least the adhesive layer 182 prior to use of the pad.

The pad connector can be inferred from the secondary cable connector 692 thereover in FIG. 7. The pad connector can include a receptacle configured for insertion of a plug of the secondary cable connector 692 therein.

Each secondary cable of the one-or-more secondary cables 672 can be operably pre-connected to a pad of the one-or-more thermoelectric pads 670 as sold. Indeed, a secondary cable can include a pad-connecting end of the secondary cable, which pad-connecting end of the secondary cable can include a secondary cable connector 692 configured to connect to the pad connector of a pad of the one-or-more thermoelectric pads 670. That said, the one-or-more secondary cables 672 need not be operably pre-connected to the one-or-more thermoelectric pads 670 as sold. Notwithstanding the foregoing, once operably connected to a remainder of the system 600, each secondary cable of the one-or-more secondary cables 672 can power and communicate with electronic circuitry including the one-or-more thermoelectric devices 690 in the thermoelectric layer 678 by way of the primary cable 606.

### Methods

Methods of hyperthermic cancer treatment vary in accordance with using the systems 100, 200, and 600. That said, a method hyperthermic cancer treatment can include at least a deploying step, a hyperthermia-inducing step, a cancer treatment-administering step, and a normothermia-inducing step. Description of such steps is set forth immediately below followed by description of steps primarily in using the system 100, 200, or 600.

The deploying step includes deploying a core temperature-determining means for determining a core temperature of the patient. The core temperature-determining means can be a core temperature-determining means set forth herein or another known core temperature-determining means. The deploying step enables the control module 102 or 602 of the system 100, 200, or 600 to adjust a core temperature of a patient to compensate for any deviance from a programmed temperature profile for the patient during the hyperthermic cancer treatment.

The hyperthermia-inducing step includes inducing hyperthermia in the patient with the system 100, 200, or 600 for hyperthermic cancer treatment.

The cancer treatment-administering step includes administering a cancer treatment including radiation therapy, chemotherapy, or immunotherapy while the patient is in a hyperthermic state, thereby complementing the cancer treatment. The cancer treatment-administering step can include maintaining hyperthermia in the patient with the system 100, 200, or 600 for at least a duration of the cancer treatment-administering step.

The normothermia-inducing step includes inducing normothermia in the patient after cancer treatment-administering step.

Adverting to the method of hyperthermic cancer treatment when using the system 100, the deploying step can further include inserting the catheter 148 into the patient. As set forth above, the catheter 148 can include the thermistor 164 in the distal portion of the catheter 148 configured for determining a core temperature of the patient.

Further to the method of hyperthermic cancer treatment when using the system 100, the method can include a thermistor-connecting step. The thermistor-connecting step includes connecting the thermistor connector 168 of the catheter 148 to the control module 102 or an intervening device (e.g., a dedicated device, a smartphone including a dedicated software application, etc.) between the thermistor connector 168 and the control module 102. The thermistor-connecting step powers the thermistor 164 and enables signals or data corresponding to microammeter-measured currents from the microammeter in series with the thermistor 164 to be communicated to the control module 102.

Further to the method of hyperthermic cancer treatment when using the system 100, the hyperthermia-inducing step can include conveying blood of the patient to the heat exchanger 140 of the system 100 by way of the primary lumen of the catheter 148. The hyperthermia-inducing step can also include conveying the blood back to the patient by way of the secondary lumen of the catheter 148. As set forth above, the conveying of the blood is made possible by pumping the blood with the peristaltic pump 142. In the hyperthermia-inducing step, the heat exchanger 140 is configured to exchange heat between the blood and the temperature-controlled system fluid conveyed to the heat exchanger 140 by way of the primary FDL 106. Specifically, the heat exchanger 140 is configured to transfer heat into the blood during the hyperthermia-inducing step.

Further to the method of hyperthermic cancer treatment when using the system 100, the cancer treatment-administering step can include intravenously administering a solution of one or more chemotherapy agents or one or more immunotherapy agents to the patient by way of the tertiary lumen of the catheter 148.

Adverting to the method of hyperthermic cancer treatment when using the system 200 or 600, the method can include a release liner-removal step. The release liner-removal step includes removing the release liner 176 of a pad of the one-or-more hydraulic pads 170 or the one-or-more thermoelectric pads 670 to reveal the adhesive layer 182. As set forth above, the release liner 176 is configured to maintain the integrity of at least the adhesive layer 182 prior to using the pad.

Further to the method of hyperthermic cancer treatment when using the system 200 or 600, the method can include a pad-placing step. The pad-placing step includes placing the pad on a portion of a body of the patient with the thermally conductive adhesive layer 182 of the multilayered pad body 174 or 674 of the pad in contact with skin S (see FIG. 5 or FIG. 7) of the patient. The pad-placing step can be performed before or after any pad-connecting step of connecting the pad to a remainder of the system 200 or 600.

Further to the method of hyperthermic cancer treatment when using the system 200, the hyperthermia-inducing step includes charging the conduit layer 178 of the pad with the supply fluid of the temperature-controlled system fluid to exchange heat between the supply fluid and the patient by thermal conduction through the adhesive layer 182. Specifically, the supply fluid is configured to transfer heat into the patient during the hyperthermia-inducing step. As set forth above, the supply fluid is provided by the hydraulic system 104 of the control module 102 by way of a combination of fluidly connected FDLs including the primary FDL 106 and the one-or-more secondary FDLs 172.

Further to the method of hyperthermic cancer treatment when using the system 600, the hyperthermia-inducing step includes applying a voltage across the one-or-more thermoelectric devices 690 of the thermoelectric layer 678 of the pad to exchange heat between the one-or-more thermoelectric devices and the patient by thermal conduction through the adhesive layer 182. Specifically, the one-or-more thermoelectric devices 690 are configured to transfer heat into the patient during the hyperthermia-inducing step. As set forth above, the voltage is applied by the control module 602 by way of at least the primary cable 606 between the pad and the control module 602.

While the method of hyperthermic cancer treatment when using the system 200 or 600 is set forth with reference to a single pad of the one-or-more hydraulic pads 170 or the one-or-more thermoelectric pads 670, it should be understood that any number of pads of the one-or-more hydraulic pads 170 or the one-or-more thermoelectric pads 670 can be used as necessary to effectuate regional or whole-body heating for the hyperthermic cancer treatment.

Further to the method of hyperthermic cancer treatment when using the system 200 or 600, the deploying step can include deploying a tympanic thermometer, a rectal thermometer, or a thermistor-tipped catheter such as the catheter 148.

Further to the method of hyperthermic cancer treatment when using the system 200 or 600, the cancer treatment-administering step can include intravenously administering a solution of one or more chemotherapy agents or one or more immunotherapy agents to the patient by way of the catheter 148 or administering the radiation therapy by way of a linear accelerator.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system for hyperthermic cancer treatment, comprising:
a heat exchanger;
a control module including at least a hydraulic system configured to provide a temperature-controlled system fluid;
a primary fluid delivery line (“FDL”) configured to convey the temperature-controlled system fluid as a supply fluid to the heat exchanger and convey a return fluid back to the hydraulic system;
an intravenous catheter including two-or-more lumens, a primary lumen of the two-or-more lumens configured to convey blood of a patient to the heat exchanger and a secondary lumen of the two-or-more lumens configured to convey the blood back to the patient; and
a peristaltic pump configured to pump the blood from the patient to the heat exchanger and pump the blood back to the patient, the system configured to induce hyperthermia in the patient for simultaneous cancer treatment.

2. The system of claim 1, wherein the two-or-more lumens include a tertiary lumen configured for intravenous administration of a solution of one or more chemotherapy agents or one or more immunotherapy agents to the patient.

3. The system of claim 1, the catheter further including a thermistor in a distal portion of the catheter configured for determining a core body temperature of the patient.

4. The system of claim 1, the catheter further including a thermistor connector in a proximal portion of the catheter, the thermistor connector being a power-and-data connector configured for a wired connection to the control module or an intervening device between the thermistor connector and the control module.

5. The system of claim 1, wherein the heat exchanger includes a thermistor configured for determining a core body temperature of the patient.

6. The system of claim 1, wherein the heat exchanger and the peristaltic pump are in a heat-exchange module separate from the control module.

7. The system of claim 1, wherein the heat exchanger and the peristaltic pump are integrated into the control module.

8. The system of claim 1, the hydraulic system further including:
a heater configured for fluid heating;
a chiller evaporator configured for fluid cooling, the heater and the chiller evaporator, together, configured to provide the temperature-controlled system fluid;
a hydraulic-system outlet configured for discharging the supply fluid from the hydraulic system; and
a hydraulic-system inlet configured for charging the hydraulic system with the return fluid to continue to produce the temperature-controlled system fluid.

9. The system of claim 1, the control module further including one or more processors, primary memory, and instructions stored in the primary memory configured to instantiate one or more processes for hyperthermic cancer treatment with the control module, the one-or-more processes including a temperature-adjusting process for adjusting a temperature of the temperature-controlled system fluid in accordance with core body-temperature measurements to compensate for any deviance from a programmed temperature profile for the patient during the hyperthermic cancer treatment.

10. A system for hyperthermic cancer treatment, comprising:
a control module including at least a hydraulic system configured to provide a temperature-controlled system fluid;
a primary fluid delivery line (“FDL”) configured to convey the temperature-controlled system fluid as a supply fluid from the hydraulic system and convey a return fluid back to the hydraulic system;
one or more hydraulic pads configured for placement on one or more portions of a body of the patient, respectively; and
a core temperature-determining means for determining a core temperature of the patient, the system configured to induce hyperthermia in the patient for simultaneous cancer treatment.

11. The system of claim 10, wherein the core temperature-determining means includes a tympanic thermometer, a rectal thermometer, a nasopharyngeal temperature probe, an esophageal temperature probe, a thermistor-tipped catheter, or a medical infrared thermometer for skin temperature adjusted with skin location and ambient temperature for core temperature.

12. The system of claim 10, each pad of the one-or-more hydraulic pads including:
a multilayered pad body including:
a conduit layer including one or more conduits configured to convey the supply fluid from the hydraulic system and convey the return fluid back to the hydraulic system; and
a thermally conductive adhesive layer over the conduit layer configured for placement on a portion of the one-or-more portions of the body of the patient;
a pad inlet connector including a pad inlet configured for charging the conduit layer with the supply fluid; and
a pad outlet connector including a pad outlet configured for discharging the return fluid from the conduit layer.

13. The system of claim 12, the pad body further including an impermeable film between the conduit layer and the adhesive layer configured to retain the supply fluid in the conduit layer.

14. The system of claim 12, wherein the adhesive layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

15. The system of claim 12, each pad of the one-or-more hydraulic pads further including a release liner over the adhesive layer in a ready-to-use state of the pad, the release liner configured to maintain integrity of at least the adhesive layer prior to use of the pad.

16. The system of claim 12, further comprising a secondary FDL for each pad of the one-or-more hydraulic pads configured to convey the supply fluid from the primary FDL and convey the return fluid back to the primary FDL, the secondary FDL split at a pad-connecting end of the secondary FDL, and the pad-connecting end of the secondary FDL including a pair of secondary FDL connectors including a secondary FDL outlet connector configured to fluidly connect to the pad inlet connector and a secondary FDL inlet connector configured to fluidly connect to the pad outlet connector.

17. The system of claim 10, the hydraulic system including:
a heater configured for fluid heating;
a chiller evaporator configured for fluid cooling, the heater and the chiller evaporator, together, configured to provide the temperature-controlled system fluid;
a hydraulic-system outlet configured for discharging the supply fluid from the hydraulic system; and
a hydraulic-system inlet configured for charging the hydraulic system with the return fluid to continue to produce the temperature-controlled system fluid.

18. The system of claim 10, the control module further including one or more processors, primary memory, and instructions stored in the primary memory configured to instantiate one or more processes for hyperthermic cancer treatment with the control module, the one-or-more processes including a temperature-adjusting process for adjusting a temperature of the temperature-controlled system fluid in accordance with core body-temperature measurements to compensate for any deviance from a programmed temperature profile for the patient during the hyperthermic cancer treatment.

19. A system for hyperthermic cancer treatment, comprising:
a control module including one or more processors, primary memory, and instructions stored in the primary memory configured to instantiate one or more processes for operating a plurality of thermoelectric devices;
a primary cable;
one or more thermoelectric pads configured for placement on one or more portions of a body of the patient, respectively, each pad of the one-or-more thermoelectric pads including one or more thermoelectric devices operable by the control module by way of at least the primary cable; and
a core temperature-determining means for determining a core temperature of the patient, the system configured to induce hyperthermia in the patient for simultaneous cancer treatment.

20. The system of claim 19, wherein the core temperature-determining means includes a tympanic thermometer, a rectal thermometer, a nasopharyngeal temperature probe, an esophageal temperature probe, a thermistor-tipped catheter, or a medical infrared thermometer for skin temperature adjusted with skin location and ambient temperature for core temperature.

21. The system of claim 19, each pad of the one-or-more thermoelectric pads including:
a multilayered pad body including:
a thermoelectric layer including the one-or-more thermoelectric devices configured to undergo a temperature change upon application of a voltage across the one-or-more thermoelectric devices; and
a thermally conductive adhesive layer over the thermoelectric layer configured for placement on a portion of the one-or-more portions of the body of the patient;
a pad connector configured for establishing an operable connection with the control module.

22. The system of claim 21, wherein the adhesive layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

23. The system of claim 22, each pad of the one-or-more thermoelectric pads further including a release liner over the adhesive layer in a ready-to-use state of the pad, the release liner configured to maintain integrity of at least the adhesive layer prior to use of the pad.

24. The system of claim 19, the one-or-more processes including a temperature-adjusting process for adjusting a temperature of the plurality of thermoelectric devices in accordance with core body-temperature measurements to compensate for any deviance from a programmed temperature profile for the patient during the hyperthermic cancer treatment.

25-38. (canceled)
